(19) United States
(12) Patent Application Publication
(10) Pub. No.: US 2003/0037386 A1
(43) Pub. Date: Feb. 27, 2003

(54) New colouring combination

(76) Inventors: David Rose, Hilden (DE); Horst Hoeffkes, Duesseldorf (DE); Bernd Meinigke, Leverkusen (DE)

Correspondence Address: HENKEL CORPORATION, 2500 RENAISSANCE BLVD, STE 200, GULPH MILLS, PA, 19406, US

(21) Appl. No.: 10/166,708

(22) Filed: Jun. 10, 2002

Related U.S. Application Data
(63) Continuation of application No. PCT/EP00/12094, filed on Dec. 1, 2000.

(30) Foreign Application Priority Data
Dec. 9, 1999 (DE) 199 59 319.1

(51) Int. Cl. A61K 7/13

(52) U.S. Cl. 8/405; 8/406; 8/415; 8/421

(57) ABSTRACT

An oxidation colorant composition and a method of coloring keratin fibers are provided. The oxidation colorant composition includes a) at least one m-phenylenediamine derivative as a secondary intermediate, and b) at least one other secondary intermediate selected from 2-amino-3-hydroxy-5-chloropyridine, 2,6-dihydroxy-3,4-dimethylpyridine, 2,6-bis-(β-hydroxyethylamino)-toluene, 2,4-dichloro-3-aminophenol, 3-amino-2-chloro-6-methylphenol, 5-amino-4-chloro-2-methylphenol, 5-(2′-hydroxyethyl)-amino-2-methylphenol, 5-amino-2-methylphenol, 2-amino-3-hydroxypyridine, 3-amino-2-methylamino-6-methoxypyridine, 2-methylresorcinol, 2,4-diaminophenoxyethanol, 1,3-bis-(2,4-diaminophenoxy)-propane, resorcinol, m-aminophenol, 3,5-diamino-2,6-dimethoxypyridine, 1,7-dihydroxynaphthalene, 2,7-dihydroxy-naphthalene, 1,5-dihydroxynaphthalene, 4-hydroxyindole and/or 6-hydroxyindole.

### CROSS-REFERENCE TO RELATED APPLICATIONS

This application is a continuation under 35 U.S.C. §365(c) and 35 U.S.C. §120 of international application PCT/EP00/12094, filed on Dec. 1, 2000, the international application not being published in English. This application also claims priority under 35 U.S.C. §119 to DE 199 59 319.1, filed on Dec. 9, 1999.

### BACKGROUND OF THE INVENTION

This invention relates to colorants containing special combinations of secondary intermediates and to the use of these colorants for coloring keratin fibers.

By virtue of their intensive colors and good fastness properties, so-called oxidation colorants play a prominent role in the coloring of keratin fibers, particularly human hair. Oxidation colorants contain oxidation dye precursors, so-called primary intermediates and secondary intermediates. The primary intermediates form the actual dyes with one another or by coupling with one or more secondary intermediates in the presence of oxidizing agents or atmospheric oxygen.

Good oxidation dye precursors are expected to satisfy above all the following requirements: they must form the required color tones with sufficient intensity and fastness during the oxidative coupling reaction. In addition, they must be readily absorbed onto the fibers with no significant differences—particularly in the case of human hair—between damaged and freshly regrown hair (levelling behavior). Finally, if they are used to color hair, they should not overly stain the scalp and, above all, should be toxicologically and dermatologically safe.

The primary intermediates normally used are primary aromatic amines containing another free or substituted hydroxy or amino group in the para position or the ortho position, diaminopyridine derivatives, heterocyclic hydrazones, 4-aminopyrazolone derivatives and 2,4,5,6-tetraaminopyrimidine and derivatives thereof. The secondary intermediates are generally m-phenylenediamine derivatives, naphthols, resorcinol and resorcinol derivatives, pyrazolones and m-aminophenols.

In general, natural color tones cannot be obtained with a primary intermediate alone or with a special secondary intermediate/primary intermediate combination. In practice, therefore, combinations of various primary intermediates and/or secondary intermediates are used. The colors obtainable with the dye combinations are expected to be fast to light, heat, perspiration, rubbing and the effect of chemical reducing agents, for example permanent wave fluids. In addition, the color obtained, for example by blonding, should be easily removable from the hair if it does not meet the individual wishes of the user and is to be taken out. Accordingly, there was a need for new primary intermediate/secondary intermediate combinations with which expressive colors covering the entire spectrum—relevant to hair colorants—from yellow, red, brown to black could be obtained and which would also represent an advance over the prior art from the toxicological perspective.

It has now surprisingly been found that hair colorants containing special derivatives of m-phenylenediamine in combination with certain other secondary intermediates satisfy the requirements hair colorants are expected to meet to a high degree.

### SUMMARY OF THE INVENTION

In a first embodiment, therefore, the present invention relates to colorants for coloring keratin fibers which, in a medium suitable for coloring, contain at least one primary intermediate and

(a) as secondary intermediate at least one m-phenylenediamine derivative corresponding to formula (I):

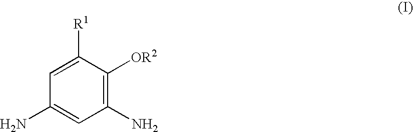

in which R¹ is a branched or unbranched C₁₋₈ alkyl group and R² is a branched or unbranched C₁₋₈ alkyl group or a phenyl group which may optionally be substituted by one or more C₁₋₄ alkyl group(s) or by one or more halogen atom(s), or a physiologically compatible salt thereof and

(b) at least one other secondary intermediate selected from 2-amino-3-hydroxy-5-chloropyridine, 2,6-dihydroxy-3,4-dimethylpyridine, 2,6-bis-(β-hydroxyethylamino)-toluene, 2,4-dichloro-3-aminophenol, 3-amino-2-chloro-6-methylphenol, 5-amino-4-chloro-2-methylphenol, 5-(2′-hydroxyethyl)-amino-2-methylphenol, 5-amino-2-methylphenol, 2-amino-3-hydroxypyridine, 3-amino-2-methylamino-6-methoxypyridine, 2-methylresorcinol, 2,4-diaminophenoxyethanol, 1,3-bis-(2,4-diaminophenoxy)-propane, resorcinol, m-aminophenol, 3,5-diamino-2,6-dimethoxypyridine, 1,7-, 2,7- and 1,5-dihydroxynaphthalene and 4-hydroxyindole and 6-hydroxyindole.

### DETAILED DESCRIPTION OF THE INVENTION

Keratin fibers in the context of the invention are pelts, wool, feathers and, in particular, human hair. Although the colorants according to the invention are mainly suitable for coloring keratin fibers, there is nothing in principle to prevent their use for other applications.

Examples of the C₁₋₈ alkyl groups mentioned as substituents in the compounds corresponding to formula (I) are the methyl, ethyl, propyl, isopropyl and butyl groups. Ethyl and methyl groups are preferred alkyl groups. According to the invention, examples of halogen atoms are F, Cl or Br atoms. Cl atoms are particularly preferred. According to the invention, physiologically compatible salts are, in particular, salts of inorganic acids, such as hydrochloric or sulfuric acid.

Compounds corresponding to formula (I) are already mentioned as oxidation dye precursors in DE 26 28 999. However, there is no reference in this document to the excellent coloring properties of the oxidation dye precursor combinations according to the invention.

The colors obtainable with these dye combinations broaden the known range of shades, particularly the various shades of red. The uniform coloring of hair fibers damaged to different extents and the excellent fastness to washing of the colors obtained with the dye combinations according to the invention are instrumental in overcoming another deficiency of the prior art well-known to the expert.

A preferred compound of formula (I) according to the invention is 1-methyl-2-methoxy-3,5-diaminobenzene or a physiologically compatible salt thereof.

Other preferred colorants according to the invention are those which contain 3-amino-2-chloro-6-methylphenol, 5-amino-4-chloro-2-methylphenol, 5-(2′-hydroxyethyl)-amino-2-methylphenol, 5-amino-2-methylphenol, 2-amino-3-hydroxypyridine, 3-amino-2-methylamino-6-methoxypyridine, 2-methylresorcinol, 2,4-diaminophenoxyethanol, 1,3-bis-(2,4-diaminophenoxy)-propane, resorcinol, m-aminophenol, 3,5-diamino-2,6-dimethoxypyridine and/or 1,5-dihydroxynaphthalene as the additional secondary intermediate (b).

Most particularly preferred secondary intermediates (b) are 3-amino-2-chloro-6-methylphenol, 5-amino-4-chloro-2-methylphenol, 2-amino-3-hydroxypyridine, 3-amino-2-methylamino-6-methoxypyridine, 2,4-diaminophenoxyethanol, 3,5-diamino-2,6-dimethoxypyridine and 1,5-dihydroxynaphthalene.

In a preferred embodiment of the invention a p-phenylenediamine derivative or a physiologically compatible salt thereof is used as the primary intermediate. Particularly preferred p-phenylenediamine derivatives correspond to formula (II):

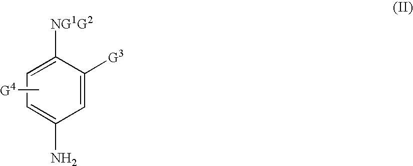

in which

G¹ stands for a hydrogen atom, a C₁₋₄ alkyl radical, a C₁₋₄ monohydroxyalkyl radical, a C2-4 polyhydroxyalkyl radical, a (C1-4)-alkoxy-(C₁₋₄)-alkyl radical, a 4′-aminophenyl radical or a C₁₋₄ alkyl radical substituted by a nitrogen-containing group, a phenyl group or a 4′-aminophenyl group;

G² stands for a hydrogen atom, a C₁₋₄ alkyl radical, a C₁₋₄ monohydroxyalkyl radical, a C₂₋₄ polyhydroxyalkyl radical, a (C1-4)-alkoxy-(C₁₋₄)-alkyl radical or a C₁₋₄ alkyl radical substituted by a nitrogen-containing group;

G³ stands for a hydrogen atom, a halogen atom, such as a chlorine, bromine, iodine or fluorine atom, a C₁₋₄ alkyl radical, a C₁₋₄ monohydroxyalkyl radical, a C₁₋₄ hydroxyalkoxy radical, a C₁₋₄ acetylaminoalkoxy radical, a C₁₋₄ mesylaminoalkoxy radical or a C1-4 carbamoylaminoalkoxy radical;

G⁴ is a hydrogen atom, a halogen atom or a C14 alkyl radical or

if G³ and G⁴ are in the ortho position to one another, they may together form a bridging α,ω-alkylenedioxo group such as, for example, an ethylenedioxy group.

Examples of the C₁₋₄ alkyl radicals mentioned as substituents in the compounds according to the invention are the methyl, ethyl, propyl, isopropyl and butyl groups. Ethyl and methyl radicals are preferred alkyl radicals. According to the invention, preferred C₁₋₄ alkoxy radicals are, for example, methoxy or ethoxy radicals. Other preferred examples of a C₁₋₄ hydroxyalkyl group are the hydroxymethyl, 2-hydroxyethyl, 3-hydroxypropyl or 4-hydroxybutyl group. A 2-hydroxyethyl group is particularly preferred. According to the invention, examples of halogen atoms are F, Cl or Br atom. Cl atoms are most particularly preferred. According to the invention, the other terms used are derived from the definitions given here. Examples of nitrogen-containing groups corresponding to formula (II) are, in particular, the amino groups, C₁₋₄ monoalkylamino groups, C₁₋₄ dialkylamino groups, C₁₋₄ trialkylammonium groups, C₁₋₄ monohydroxyalkylamino groups, imidazolinium and ammonium.

Particularly preferred p-phenylenediamines corresponding to formula (II) are selected from p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)-aniline, N,N-bis-(p-hydroxyethyl)-p-phenylenediamine, 4-N,N-bis-(β-hydroxyethyl)-amino-2-methylaniline, 4-N,N-bis-(β-hydroxyethyl)-amino-2-chloroaniline, 2-(β-hydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl-β-hydroxyethyl)-p-phenylene-diamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylenediamine and 5,8-diaminobenzo-1,4-dioxane and physiologically compatible salts thereof.

According to the invention, most particularly preferred p-phenylenediamine derivatives corresponding to formula (II) are p-phenylenediamine, p-toluylenediamine, 2-(β-hydroxyethyl)-p-phenylene-diamine and N,N-bis-(2-hydroxyethyl)-p-phenylenediamine and physiologically compatible salts thereof.

In another preferred embodiment of the invention, compounds containing at least two aromatic nuclei substituted by amino and/or hydroxyl groups may be used as primary intermediates.

The binuclear primary intermediates which may be used in the coloring compositions according to the invention include in particular compounds corresponding to formula (III) and physiologically compatible salts thereof:

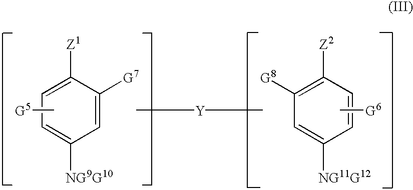

in which

Z¹ and Z² independently of one another stand for a hydroxyl or NH₂ radical optionally substituted by a C₁₋₄ alkyl radical, by a C₁₋₄ hydroxyalkyl radical and/or by a bridging group Y,

the bridging group Y is a C₁₋₁₄ alkylene group such as, for example, a linear or branched alkylene chain or an alkylene ring which may be interrupted or terminated by one or more nitrogen-containing groups and/or one or more hetero atoms, such as oxygen, sulfur or nitrogen atoms, and may optionally be substituted by one or more hydroxyl or C₁₋₈ alkoxy radicals,

G⁵ and G⁶ independently of one another stand for a hydrogen or halogen atom, a C₁₋₄ alkyl radical, a C₁₋₄ monohydroxyalkyl radical, a C₂₋₄ polyhydroxyalkyl radical, a C₁₋₄ aminoalkyl radical or a direct bond to the bridging group Y,

G⁷, G⁸, G⁹, G¹⁰,G¹¹ and G¹² independently of one another stand for a hydrogen atom, a direct bond to the bridging group Y or a C₁₄ alkyl radical,

with the proviso that the compounds of formula (III) contain only one bridging group Y per molecule.

According to the invention, the substituents used in formula (III) are as defined in the foregoing.

Preferred examples of nitrogen-containing groups corresponding to formula (III) are amino radicals, C₁₋₄ monoalkylamino radicals, C₁₋₄ dialkylamino radicals, C₁₋₄ trialkyl ammonium radicals, C₁₋₄ monohydroxyalkylamino radicals, imidazolinium and ammonium.

Preferred binuclear primary intermediates corresponding to formula (III) are, in particular, N,N′-bis-(β-hydroxyethyl)-N,N′-bis-(4′-aminophenyl)-1,3-diaminopropanol, N ,N′-bis-(β-hydroxyethyl)-N,N′-bis-(4′-aminophenyl)-ethylenediamine, N,N′-bis-(4-aminophenyl)-tetramethylene diamine, N,N′-bis-(β-hydroxyethyl)-N,N′-bis-(4′-aminophenyl)-tetramethylene diamine, N,N′-bis-(4-methylaminophenyl)-tetramethylene diamine, N,N′-bis-(ethyl)-N,N′-bis-(4′-amino-3′-methylphenyl)-ethylenediamine, 1,8-bis-(2,5-diamino-phenoxy)-3,5-dioxaoctane, bis-(2-hydroxy-5-aminophenyl)-methane, 1,4-bis-(4-aminophenyl)-diazacycloheptane and 1,10-bis-(2,5-diaminophenyl)-1,4,7,10-tetraoxadecane and physiologically compatible salts thereof.

Most particularly preferred binuclear primary intermediates corresponding to formula (III) are N,N′-bis-(β-hydroxyethyl)-N,N′-bis-(4′-aminophenyl)-1,3-diaminopropanol, bis-(2-hydroxy-5-aminophenyl)-methane, N,N′-bis-(4-aminophenyl)-1,4-diazacycloheptane and 1,10-bis-(2,5-diaminophenyl)-1,4,7,10-tetraoxadecane or a physiologically compatible salt thereof. Of these, bis-(2-hydroxy-5-aminophenyl)-methane is quite most particularly preferred.

In another preferred embodiment of the invention, a p-aminophenol derivative or a physiologically compatible salt thereof is used as the primary intermediate. Particularly preferred p-aminophenol derivatives correspond to formula (IV):

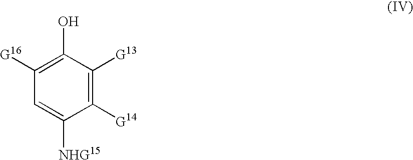

in which

G¹³ stands for a hydrogen atom, a halogen atom, a C14 alkyl radical, a C₁₋₄ monohydroxyalkyl radical, a (C₁₋₄)-alkoxy-(C₁₋₄)-alkyl radical, a C₁₋₄ aminoalkyl radical, a hydroxy-(C₁₋₄)-alkylamino radical, a C₁₋₄ hydroxyalkyoxy radical, a C₁₋₄ hydroxyalkyl-(C₁₋₄)-aminoalkyl radical or a (di-C₁₋₄-alkylamino)-(C₁₋₄)-alkyl radical,

G¹⁴ stands for a hydrogen atom or a halogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical, a C₁₋₄ aminoalkyl radical or a C₁₋₄ cyanoalkyl radical,

G¹⁵ stands for hydrogen, a C₁₋₄ alkyl radical, a C₁₋₄ monohydroxyalkyl radical, a C₂₋₄ polyhydroxyalkyl radical, a phenyl radical or a benzyl radical and

G¹⁶ stands for hydrogen or a halogen atom.

According to the invention, the substituents used in formula (IV) are defined as in the foregoing.

Preferred p-aminophenols corresponding to formula (IV) are, in particular, p-aminophenol, N-methyl-p-aminophenol, 4-amino-3-methyl-phenol, 4-amino-3-fluorophenol, 2-hydroxymethylamino-4-aminophenol, 4-amino-3-hydroxymethylphenol, 4-amino-2-(2-hydroxyethoxy)-phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)-phenol, 4-amino-2-fluorophenol, 4-amino-2-chlorophenol, 2,6-dichloro-4-aminophenol, 4-amino-2-((diethylamino)-methyl)phenol and physiologically compatible salts thereof.

Most particularly preferred compounds corresponding to formula (IV) are p-aminophenol, 4-amino-3-methylphenol, 4-amino-2-aminomethyl-phenol and 4-amino-2-((diethylamino)methyl)phenol.

The primary intermediate may also be selected from o-aminophenol and its derivatives such as, for example, 2-amino-4-methylphenol or 2-amino-4-chlorophenol.

The primary intermediate may also be selected from heterocyclic primary intermediates such as, for example, pyridine, pyrimidine, pyrazole, pyrazole/pyrimidine derivatives and physiologically compatible salts thereof.

Preferred pyridine derivatives are, in particular, the compounds described in GB 1,026,978 and GB 1,153,196, such as 2,5-diaminopridine, 2-(4-methoxyphenyl)-amino-3-aminopyridine, 2,3-diamino-6-methoxy-pyridine, 2-(β-methoxyethyl)-amino-3-amino-6-methoxypyridine and 3,4-diaminopyridine.

Preferred pyrimidine derivatives are, in particular, the compounds described in DE 2359399, JP 02019576 A2 and WO 96/15765, such as 2,4,5,6-tetraaminopyrimidine, 4-hydroxy-2,5,6-triaminopyrimidine, 2-hydroxy-4,5,6-triaminopyrimidine, 2-dimethylamino-4,5,6-triamino-pyrimidine, 2,4-dihydroxy-5,6-diaminopyrimidine and 2,5,6-triaminopyridine.

Preferred pyrazole derivatives are, in particular, the compounds described in DE 3843892, DE 4133957, WO 94/08969, WO 94/08970, EP 740931 and DE 19543988, such as 4,5-diamino-1-methylpyrazole, 4,5-diamino-1-(β-hydroxyethyl)-pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methyl pyrazole, 4,5-diamino-3-tert.butyl-1-methylpyrazole, 4,5-diamino-1-tert.butyl-3-methylpyrazole, 4,5-diamino-1-(β-hydroxyethyl)-3-methyl-pyrazole, 4,5-diamino-1-ethyl-3-methylpyrazole, 4,5-diamino-1-ethyl-3-(4′-methoxyphenyl)-pyrazole, 4,5-diamino-1-ethyl-3-hydroxymethylpyrazole, 4,5-diamino-3-hydroxymethyl-1-methylpyrazole, 4,5-diamino-3-hydroxy-methyl-1-isopropylpyrazole, 4,5-diamino-3-methyl-1-isopropylpyrazole, 4-amino-5-(2′-aminoethyl)-amino-1,3-dimethylpyrazole, 3,4,5-triamino-pyrazole, 1-methyl-3,4,5-triaminopyrazole, 3,5-diamino-1-methyl-4-methyl-aminopyrazole and 3,5-diamino-4-(β-hydroxyethyl)-amino-1-methyl-pyrazole.

Preferred pyrazole-pyrimidine derivatives are, in particular, the derivatives of pyrazole-[1,5-a]-pyrimidine corresponding to formula (V) below and tautomeric forms thereof where a tautomeric equilibrium exists:

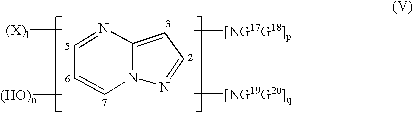

in which

G¹⁷, G¹⁸, G¹⁹ and G²⁰ independently of one another stand for a hydrogen atom, a C₁₋₄ alkyl radical, an aryl radical, a C₁₋₄ hydroxyalkyl radical, a C₂₋₄ polyhydroxyalkyl radical, a (C₁₋₄)-alkoxy-(C₁₋₄⁾-alkyl radical, a C₁₋₄ aminoalkyl radical which may optionally be protected by an acetylureide or sulfonyl radical, a (C₁₋₄)-alkylamino-(C₁₋₄)-alkyl radical, a di[(C₁₄)-alkyl]-(C₁₋₄)-aminoalkyl radical, the dialkyl radicals optionally forming a carbon cycle or a heterocycle with 5 or 6 links, a C₁₋₄ hydroxyalkyl or a di-(C₁₋₄)-[hydroxyalkyl](C₁₋₄)-aminoalkyl radical;

the X radicals independently of one another stand for a hydrogen atom, a C₁₋₄ alkyl radical, an aryl radical, a C₁₋₄ hydroxyalkyl radical, a C₂₋₄ polyhydroxyalkyl radical, a C₁₋₄ aminoalkyl radical, a (C₁₋₄)-alkylamino-(C₁₋₄)-alkyl radical, a di[(C₁₋₄)-alkyl]-(C₁₋₄)-aminoalkyl radical, the dialkyl radicals optionally forming a carbon cycle or a heterocycle with 5 or 6 links, a C₁₋₄ hydroxyalkyl or a di-(C₁₋₄)-[hydroxyalkyl]-(C₁₋₄)-aminoalkyl radical, an amino radical, a C₁₋₄ alkyl or a di-(C₁₋₄ hydroxyalkyl)-amino radical, a halogen atom, a carboxylic acid group or a sulfonic acid group,

i has the value 0, 1, 2 or 3,

p has the value 0 or 1,

q has the value 0 or 1 and

n has the value 0 or 1,

with the proviso that

the sum of p+q is not 0,

where p+q=2, n has the value 0 and the groups NG¹⁷G¹⁸ and NG¹⁹G²⁰ occupy the (2,3); (5,6); (6,7); (3,5) or (3,7) positions;

where p+q=1, n has the value 1 and the groups NG¹⁷G¹⁸ (or NG¹⁹G²⁰) and the group OH occupy the (2,3); (5,6); (6,7); (3,5) or (3,7) positions.

The substituents used in formula (V) are as defined in the foregoing.

If the pyrazole-[1,5-a]-pyrimidine corresponding to formula (V) above contains a hydroxy group in one of the positions 2, 5 or 7 of the ring system, a tautomeric equilibrium exists as illustrated, for example, in the following scheme:

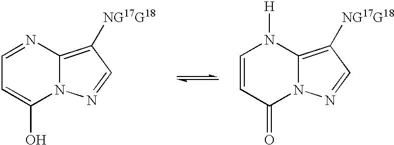

Among the pyrazole-[1,5-a]-pyrimidines corresponding to formula (V) above, the following may be particularly mentioned:

pyrazole-[1,5-a]-pyrimidine-3,7-diamine;

2,5-dimethylpyrazole-[1,5-a]-pyrimidine-3,7-diamine;

pyrazole-[1,5-a]-pyrimidine-3,5-diamine;

2,7-dimethylpyrazole-[1,5-a]-pyrimidine-3,5-diamine;

3-aminopyrazole-[1,5-a]-pyrimidin-7-ol;

3-aminopyrazole-[1,5-a]-pyrimidin-5-ol;

2-(3-aminopyrazole-[1,5-a]-pyrimidin-7-ylamino)-ethanol;

2-(7-aminopyrazole-[1,5-a]-pyrimidin-3-ylamino)-ethanol;

2-[(3-aminopyrazole-[1,5-a]-pyrimidin-7-yl)-(2-hydroxyethyl)-amino]-ethanol;

2-[(7-aminopyrazole-[1,5-a]-pyrimid in-3-yl)-(2-hydroxyethyl)-amino]-ethanol;

5,6-dimethylpyrazole-[1,5-a]-pyrimidine-3,7-diamine;

2,6-dimethylpyrazole-[1,5-a]-pyrimidine-3,7-diamine;

2,5,N7,N7-tetramethylpyrazole-[1,5-a]-pyrimidine-3,7-diamine;

and physiologically compatible salts thereof and tautomeric forms thereof where a tautomeric equilibrium exists.

The pyrazole-[1,5-a]-pyrimidines corresponding to formula (V) above may be prepared by cyclization from an aminopyrazole or from hydrazine, as described in the literature.

In addition, the colorants according to the invention may contain one or more other secondary intermediates such as, for example,

m-aminophenol derivatives thereof such as, for example, 2-hydroxy-4-aminophenoxyethanol, 2,6-dimethyl-3-aminophenol, 3-trifluoroacetylamino-2-chloro-6-methylphenol, 5-amino-4-methoxy-2-methylphenol, 3-(diethylamino)-phenol, N-cyclopentyl-3-aminophenol, 1,3-dihydroxy-5-(methylamino)-benzene and 3-(ethylamino)-4-methylphenol,

o-aminophenol and derivatives thereof,

m-diaminobenzene and derivatives thereof such as, for example, 1-methoxy-2-amino-4-(2′-hydroxyethylamino)-benzene, 1,3-bis-(2,4-diaminophenyl)-propane and 1-amino-3-bis-(2′-hydroxyethyl)-aminobenzene,

-o-diaminobenzene and derivatives thereof such as, for example, 3,4-diaminobenzoic acid and 2,3-diamino-1-methylbenzene,

di- and trihydroxybenzene derivatives such as, for example, resorcinol monomethyl ether, 5-methyl resorcinol, 2,5-dimethyl resorcinol, 2-chlororesorcinol, 4-chlororesorcinol, pyrogallol and 1,2,4-trihydroxybenzene,

pyridine derivatives such as, for example, 2,6-dihydroxypyridine, 2,6-dihydroxy-4-methylpyridine, 2,6-diaminopyridine and 2,3-diamino-6-methoxypyridine,

naphthalene derivatives such as, for example, 1-naphthol, 2-methyl-1-naphthol, 2-hydroxymethyl-1-naphthol, 2-hydroxyethyl-1-naphthol, 1,6-dihdroxynaphthalene, 1,8-dihdroxynaphthalene and 2,3-dihdroxynaphthalene,

morpholine derivatives such as, for example, 6-hydroxybenzomorpholine and 6-aminobenzomorpholine,

quinoxaline derivatives such as, for example, 6-methyl-1,2,3,4-tetrahydroquinoxaline,

pyrazole derivatives such as, for example, 1-phenyl-3-methylpyrazol-5-one,

indole derivatives such as, for example, 7-hydroxyindole,

pyrimidine derivatives such as, for example, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine or

methylenedioxybenzene derivatives such as, for example, 1-hydroxy-3,4-methylenedioxybenzene, 1-amino-3,4-methylene-dioxybenzene and 1-(2′-hydroxyethyl)-amino-3,4-methylene-dioxybenzene.

Particularly preferred other secondary intermediates are 1-naphthol, 4-chlororesorcinol, 5-methyl resorcinol and 2,5-dimethyl resorcinol.

The primary and secondary intermediates are normally used in free form. In the case of compounds containing amino groups, however, it may be preferred to use them in salt form, more particularly in the form of the hydrochlorides and sulfates.

The hair colorants according to the invention contain both the primary intermediates and the secondary intermediates in a quantity of preferably 0.005 to 20% by weight and more preferably in a quantity of 0.1 to 5% by weight, based on the oxidation colorant as a whole. The primary intermediates and secondary intermediates are generally used in a substantially equimolar ratio to one another. Although it has proved to be of advantage to use the primary and secondary intermediates in an equimolar ratio, there is no disadvantage in using individual oxidation dye precursors in a certain excess so that primary intermediates and secondary intermediates may be present in a molar ratio of 1:0.5 to 1:3 and, more particularly, 1:1 to 1:2.

Substantive dyes are typically nitrophenylenediamines, nitroaminophenols, azo dyes, anthraquinones or indophenols. Preferred substantive dyes are the compounds known under the International names or commercial names of HC Yellow 2, HC Yellow 4, HC Yellow 5, HC Yellow 6, Basic Yellow 57, HC Orange 1, Disperse Orange 3, HC Red 1, HC Red 3, HC Red 13, HC Red BN, Basic Red 76, HC Blue 2, HC Blue 12, Disperse Blue 3, Basic Blue 7, Basic Blue 99, HC Violet 1, Disperse Violet 1, Disperse Violet 4, Basic Violet 14, Acid Violet 43, Disperse Black 9, Acid Black 52, Basic Brown 16 and Basic Brown 17 and also 1,4-bis-(β-hydroxyethyl)-amino-2-nitrobenzene, 3-nitro-4-(β-hydroxyethyl)-amino-phenol, 4-amino-2-nitrodiphenylamine-2′-carboxylic acid, 6-nitro-1,2,3,4-tetrahydroquinoxaline, 2-hydroxy-1 ,4-naphthoquinone, hydroxyethyl-2-nitrotoluidine, picramic acid, 2-amino-6-chloro-4-nitrophenol, 4-ethylamino-3-nitrobenzoic acid and 2-chloro-6-ethylamino-1-hydroxy-4-nitrobenzene.

The colorants according to the invention in this embodiment preferably contain the substantive dyes in a quantity of 0.01 to 20% by weight, based on the colorant as a whole.

The preparations according to the invention may also contain naturally occurring dyes such as, for example, henna red, henna neutral, henna black, camomile blossom, sandalwood, black tea, black alder bark, sage, logwood, madder root, catechu, sedre and alkanet.

Other dye components present in the colorants according to the invention include indoles and indolines and physiologically compatible salts thereof. Preferred examples are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-aminoindole and 4-aminoindole. Other preferred examples are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, 6-hydroxy-indoline, 6-aminoindoline and 4-aminoindoline.

The oxidation dye precursors or the substantive dyes do not have to be single compounds. On the contrary, other components may be present in small quantities in the hair colorants according to the invention due to the processes used to produce the individual dyes providing these other components do not adversely affect the coloring result or have to be ruled out for other reasons, for example toxicological reasons.

In another embodiment of the present invention, the hair colorants additionally contain at least one dye of the reactive carbonyl compound type selected from the group of aromatic, heteroaromatic or unsaturated aldehydes or ketones, dialdehydes or diketones or acetals, semiaminals or imine derivatives of such reactive carbonyl compounds.

Hair dyes of the reactive carbonyl compound type have been known for some time. Suitable compounds of the aromatic aldehyde type are described, for example, in DE 196 30 274 Al and in DE 196 30 275 A1. Suitable compounds are, for example, 2-hydroxybenzaldehyde, 4-hydroxy-3-methoxybenzaldehyde (vanillin) and 4-hydroxy-3-methoxycinnam-aldehyde (coniferyl aldehyde).

Suitable compounds of the heteroaromatic aldehyde type are described, for example, in DE 197 17 280 A1. Particularly suitable dyes are, for example, trans-β-(2-furyl)-acrolein, 1-methylindole-3-aldehyde, 2-(1,3,3-trimethyl-2-indolinylidene)-acetaldehyde or antipyrine-4-aldehyde. Special products of this type containing a pyridinium group are described in German patent application DE 197 45 356.2, for example the eminently suitable 4-formyl-1-methylpyridinium benzenesulfonate and 4-formyl-1-methylquinolinium methane sulfonate or methyl sulfate.

Suitable dyes of the unsaturated aldehyde type are described, for example, in DE 197 17 224 A1. Glutaconaldehyde in the form of its salts, for example its alkali metal or tetrabutylammonium salt, or 2-chloro-3-hydroxymethylene-1-cyclohexene-1-aldehyde are particularly suitable for the purposes of the present invention.

Dialdehydes and diketones and derivatives thereof suitable as dyes in accordance with the invention are, for example, alicyclic and cyclic 1,2- and 1,3-dicarbonyl compounds, such as isatin, ninhydrin, alloxane, isobarbituric acid, p- and o-quinones, 1,3-indanediones and derivatives thereof. Such dyes can be found, for example, in DE 43 35 627 A1. Suitable compounds are, for example, malondialdehyde, preferably in the form of its dimethyl acetal, 2-nitro-1,3-indanedione or 2-acetyl-1,3-cyclohexanedione.

Diketones suitable for the purposes of the invention also include cyclic dicarbonyl compounds such as, for example, isatin and derivatives thereof as described, for example, in DE 44 09 143 A1. According to the invention, preferred isatins/derivatives are, for example, isatin, isatic acid potassium salt, isatin-5-sulfonic acid potassium salt, N-allyl isatin, 1-piperidinomethyl isatin, 1-hydroxymethyl isatin and 1-diethylaminomethyl-isatin.

Another suitable cyclic dicarbonyl compound is, for example, dehydroascorbic acid of which the suitability as a hair dye is known from DE 197 45 354. Finally, acetals, imine derivatives and semiaminals of the reactive carbonyl compounds mentioned are also suitable. Compounds such as these are obtained by reaction of the carboxyl group with primary alcohols or amines and optionally elimination of water.

The group of merocyanine and azomethine dyes are obtained from the unsaturated dialdehydes and diketones. Suitable imine derivatives of glutacondialdehyde are, for example, the mono-N-methyl aniline derivative of glutaconaldehyde (5-N-methylanilinopentadienal) or N-(5-anilino-2,4-pentadien-1-ylidene)-anilinium chloride. Another suitable vinylogous cyanine dye is 7-dimethylamino-2,4,6-heptatrienylidene dimethylammonium perchlorate. Such compounds are known as hair colorant components, for example from DE 197 17223 A1.

Many of the reactive carbonyl compounds mentioned color keratin-containing fibers particularly intensively and bring out various shades only when combined with one or more color-intensifying compounds containing a primary or secondary amino- or hydroxy group selected from the group of amino acids and peptides, aromatic amines, phenols, aminophenols and nitrogen-containing heterocycles.

In many cases, deeper (darker) shades are also obtained.

Suitable amino acids are, for example, the naturally occurring and synthetic amino acids, for example arginine, histidine, phenylalanine, dihydroxyphenylalanine, ornithine, lysine. Suitable peptides are, above all, oligo- and polypeptides which have adequate solubility in water in the preparations according to the invention for reducing keratin. Examples include glutathione or the oligopeptides present in the hydrolyzates of collagen, keratin, elastin, casein, vegetable proteins, such as soya protein, wheat gluten or almond protein.

Suitable aromatic amines and aminophenols are N,N-dimethyl-, N,N-diethyl-, N-(2-hydroxyethyl)-N-ethyl-, N, N-bis-(2-hydroxyethyl)-, N-(2-methoxyethyl)-, 2-chloro-, 2,3-, 2,4- and 2,5-dichloro-p-phenylenediamine, 2,5-dihydroxy-4-morpholinoaniline dihydrobromide, 2-, 3-, 4-aminophenol, 2-aminomethyl-4-aminophenol, 2-hydroxymethyl-4-aminophenol, o- and p-phenylenediamine, o- and m-toluylenediamine, 2,5-diaminophenol, -toluene and -phenethol, 4-amino-3-methylphenol, 2-(2,5-diaminophenyl)-ethanol, 2,4-diaminophenoxyethanol, 2-(2,5-diaminophenoxy)-ethanol, 4-methylamino-, 3-amino-4-(2′-hydroxyethyloxy)-, 3,4-methylenediamino- and 3,4-methylenedioxyaniline, 3-amino-2,4-dichloro-, 4-methylamino-, 2-methyl-5-amino-, 3-methyl-4-amino-, 2-methyl-5-(2-hydroxyethylamino)-, 6-methyl-3-amino-2-chloro-, 2-methyl-5-amino-4-chloro-, 3,4-methylenedioxy-, 5-(2-hydroxyethylamino)-4-methoxy-2-methylphenol, 4-amino-2-hydroxymethylphenol, 1,3-diamino-2,4-dimethoxybenzene, 2-, 3-, 4-aminobenzoic acid, -phenylacetic acid, 2,3-, 2,4-, 2,5-, 3,4-, 3,5-diaminobenzoic acid, 4-, 5-aminosalicylic acid, 3-amino-4-hydroxy-, 4-amino-3-hydroxybenzoic acid, 2-, 3-, 4-aminobenzenesulfonic acid, 3-amino-4-hydroxybenzenesulfonic acid, 4-amino-3-hydroxynaphthalene-1-sulfonic acid, 6-amino-7-hydroxynaphthalene-2-sulfonic acid, 7-amino-4-hydroxy-naphthalene-2-sulfonic acid, 4-amino-5-hydroxynaphthalene-2,7-disulfonic acid, 3-amino-2-naphthoic acid, 3-aminophthalic acid, 5-aminoisophthalic acid, 1,3,5-, 1,2,4-triaminobenzene, 1,2,4,5-tetraaminobenzene, 2,4,5-tri-aminophenol, pentaaminobenzene, hexaaminobenzene, 2,4,6-triaminoresorcinol, 4,5-diaminopyrocatechol, 4,6-diaminopyrogallol, 3,5-diamino-4-hydroxypyrocatechol, aromatic anilines and phenols containing another aromatic radical corresponding to formula (VI):

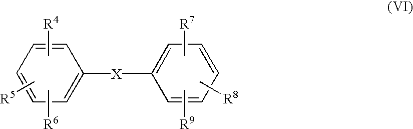

in which R⁴ is a hydroxy group or an amino group which may be substituted by C₁₋₄ alkyl, C₁₋₄ hydroxyalkyl or C₁₋₄-alkoxy-C₁₋₄-alkyl groups,

R⁵, R⁶, R⁷, R⁸ and R⁹ represent hydrogen, a hydroxy group or an amino group, which may be substituted by C₁₋₄ alkyl, C₁₋₄ hydroxyalkyl; C₁₋₄ aminoalkyl or C₁₋₄-alkoxy-C₁₋₄-alkyl groups, a carboxylic or sulfonic acid group and

X is a direct bond, a saturated or unsaturated, optionally hydroxy-substituted carbon chain containing 1 to 4 carbon atoms, a carbonyl, sulfonyl or imino group, an oxygen or sulfur atom or a group corresponding to formula (VII):

Z-(CH₂—Y—CH₂-Z′)ₒ (VII)

in which

Y is a direct bond, a CH₂ or CHOH group,

Z and Z′ independently of one another represent an oxygen atom, an NR¹⁰ group, where R¹⁰ is hydrogen, a C₁₋₄ alkyl or a hydroxy-C₁₋₄-alkyl group, the group O—(CH₂)ₚ-NH or NH—(CH₂)_{p′}-O, where p and p′=2 or 3, and o is a number of 1 to 4,

such as for example 4,4′-diaminostilbene, 4,4′-diaminostilbene-2,2′-disulfonic acid monosodium or disodium salt, 4,4′-diaminodiphenyl methane, -sulfide, -sulfoxide, -amine, 4,4′-diaminodiphenylamine-2-sulfonic acid, 4,4′-diaminobenzophenone, -diphenyl ether, 3,3′,4,4′-tetraaminodiphenyl, 3,3′4,4′-tetraaminobenzophenone, 1,3-bis-(2,4-diaminophenoxy)-propane, 1,8-bis-(2,5-diaminophenoxy)-3,6-dioxaoctane, 1,3-bis-(4-aminophenyl-amino)-propane, -2-propanol, 1,3-bis-[N-(4-aminophenyl)-2-hydroxyethyl-amino]-2-propanol, N,N-bis-[2-(4-aminophenoxy)-ethyl]-methylamine, N-phenyl-1,4-phenylenediamine.

The compounds mentioned above may be used both in free form and in the form of their physiologically compatible salts, more especially as salts of inorganic acids, such as hydrochloric acid or sulfuric acid.

Suitable phenols are, for example, 2-; 3- or 4-methoxyphenol, 3-dimethylaminophenol, 2-(2-hydroxyethyl)- and 3,4-methylenedioxyphenol, resorcinol and 2-, 4- and 5-methylresorcinol, 2- and 4-chlororesorcinol, 2,5-dimethylresorcinol, pyrocatechol, hydroquinone, pyrogallol, phloroglucinol, hydroxyhydroquinone, 2,4- or 3,4-dihydroxybenzoic or phenylacetic acid, gallic acid, 2,4,6-trihydroxybenzoic acid or 2,4,5-trihydroxyacetophenone, 1-naphthol, 1,5-, 2,3- and 2,7-dihydroxynaphthalene, 6-dimethylamino-4-hydroxy-2-naphthalenesulfonic acid or 3,6-dihydroxy-2,7-naphthalene-disulfonic acid.

Suitable nitrogen-containing heterocyclic compounds are, for example, 2-, 3-, 4-amino-, 2-amino-3-hydroxy-, 2,6-diamino-, 2,5-diamino-, 2,3-diamino-, 2-dimethylamino-5-amino-, 2-methylamino-3-amino-6-methoxy-, 2,3-diamino-6-methoxy-, 2,6-dimethoxy-3,5-diamino-, 2,4,5-triamino- and 2,6-dihydroxy-3,4-dimethyl pyridine, 2,4-dihydroxy-5,6-diamino-, 4,5,6-tri-amino-, 4-hydroxy-2,5,6-triamino-, 2-hydroxy-4,5,6-triamino-, 2,4,5,6-tetra-amino-, 2-methylamino-4,5,6-triamino-, 2,4-, 4,5-diamino-, 2-amino-4-methoxy-6-methyl pyrimidine, 3-amino-, 3-amino-5-hydroxy- and 3,5-diaminopyrazole, 2-, 3-, 8-aminoquinoline, 4-aminoquinaldine, 2-, 6-aminonicotinic acid, 5-aminoisoquinoline, 5-, 6-aminoindazole, 5- and 7-aminobenzimidazole and -benzothiazole, 2,5-dihydroxy-4-morpholinoaniline and indole and indoline derivatives, such as 4-, 5-, 6-, 7-aminoindole, 5,6-dihydroxyindole, 5,6-dihydroxyindoline and 4-hydroxyindoline. The compounds mentioned above may be used both in free form and in the form of their physiologically compatible salts, for example as salts of inorganic acids, such as hydrochloric acid or sulfuric acid.

These coloring systems may be further strengthened by suitable nitrogen-containing heterocycles such as, for example, piperidine, piperidine-2-, -3- or -4-carboxylic acid, pyridine, 2-, 3- or 4-hydroxypyridine, imidazole, 1-methylimidazole, histidine, pyrrolidine, pyrrolidone, pyrrolidone-5-carboxylic acid, pyrazole, 1,2,4-triazole, piperazine and physiologically compatible salts thereof.

So far as the dyes suitable for use in the hair colorants and tinting compositions according to the invention are concerned, reference is also expressly made to the work by Ch. Zviak, The Science of Hair Care, Chapter 7 (pages 248-250; substantive dyes) and Chapter 8, pages 264-267; oxidation dye precursors), published as Volume 7 of the Series “Dermatology” (Ed.: Ch. Culnan and H. Maibach), Marcel Dekker Inc., New York/Basel, 1986, and to the “Europäische Inventar der Kosmetik-Rohstoffe” published by the Europäische Gemeinschaft and available on floppy disk from the Bundesverband Deutscher Industrie- und Handelsunternehmen für Arzneimittel, Reformwaren und Körperpflegemittel d. V., Mannheim.

The present invention also relates to the use of the colorants described in the foregoing for coloring keratinous fibers.

To produce the colorants according to the invention, the oxidation dye precursors may be incorporated in a suitable water-containing carrier. For coloring hair, such carriers are, for example, creams, emulsions, gels or even surfactant-containing foaming solutions, for example shampoos, foam aerosols or other preparations suitable for application to the hair.

The colorants according to the invention may also contain any of the known active substances, additives and auxiliaries typical of such formulations. In many cases, the colorants contain at least one surfactant, both anionic and zwitterionic, ampholytic, nonionic and cationic surfactants being suitable in principle. In many cases, however, it has been found to be of advantage to select the surfactants from anionic, zwitterionic or nonionic surfactants.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide and hydroxyl groups may also be present in the molecule. The following are examples of suitable anionic surfactants—in the form of the sodium, potassium and ammonium salts and the mono-, di- and trialkanol-ammonium salts containing 2 or 3 carbon atoms in the alkanol group:

linear fatty acids containing 10 to 22 carbon atoms (soaps),

ether carboxylic acids corresponding to the formula R—O—(CH₂—CH₂O)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the INCI name of Cocamidopropyl Betaine.

Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl amino-propionate, cocoacyl aminoethyl aminopropionate and C₁₂₋₁₈ acyl sarcosine.

Nonionic surfactants contain, for example, a polyol group, a polyalkylene glycol ether group or a combination of polyol and polyglycol ether groups as the hydrophilic group. Examples of such compounds are

products of the addition of 2 to 30 moles of ethylene oxide and/or 0 to 5 moles of propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of products of the addition of 1 to 30 moles of ethylene oxide onto glycerol,

C₈₋₂₂ alkyl mono- and oligoglycosides and ethoxylated analogs thereof,

products of the addition of 5 to 60 moles of ethylene oxide onto castor oil and hydrogenated castor oil,

products of the addition of ethylene oxide onto sorbitan fatty acid esters and

products of the addition of ethylene oxide onto fatty acid alkanolamides.

Examples of cationic surfactants suitable for use in the hair treatment preparations according to the invention are, in particular, quaternary ammonium compounds. Preferred quaternary ammonium compounds are ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. Other cationic surfactants suitable for use in accordance with the invention are the quaternized protein hydrolyzates.

Also suitable for the purposes of the invention are cationic silicone oils such as, for example, the commercially available products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80)

Alkyl amidoamines, particularly fatty acid amidoamines, such as the stearyl amidopropyl dimethyl amine obtainable as Tego Amid®S 18, are distinguished not only by their favorable conditioning effect, but also and in particular by their ready biodegradability.

Quaternary ester compounds, so-called “esterquats”, such as the methyl hydroxyalkyl dialkoyloxyalkyl ammonium methosulfates marketed under the trade name of Stepantex® and the products marketed under the trade name of Dehyquart®, such as Dehyquart® AU-46, are also readily biodegradable.

One example of a quaternary sugar derivative suitable for use as a cationic surfactant is the commercially available product Glucquat®100 (INCI name: Lauryl Methyl Gluceth-10Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

The hair treatment preparations according to the invention preferably may also contain a conditioning agent selected from the group consisting of cationic surfactants, cationic polymers, alkyl amidoamines, paraffin oils, vegetable oils and synthetic oils.

Cationic polymers can be preferred conditioning agents. These are generally polymers containing a quaternary nitrogen atom, for example in the form of an ammonium group. The following are examples of preferred cationic polymers:

Quaternized cellulose derivatives commercially available under the names of Celquat® and Polymer JR®. The compounds Celquat® H 100, Celquat® L 200 and Polymer JR®400 are preferred quaternized cellulose derivatives.

Polymeric dimethyl diallyl ammonium salts and copolymers thereof with acrylic acid and with esters and amides of acrylic acid and methacrylic acid. The products commercially available under the names of Merquat®100 (poly(dimethyl diallyl ammonium chloride)), Merquat®550 (dimethyl diallyl ammonium chloride/acrylamide copolymer) and Merquat® 280 (dimethyl diallyl ammonium chloride/acrylic acid copolymer) are examples of such cationic polymers.

Copolymers of vinyl pyrrolidone with quaternized derivatives of dialkylaminoacrylate and methacrylate, such as vinyl pyrrolidone/dimethylaminomethacrylate copolymers quaternized, for example, with diethyl sulfate. Compounds such as these are commercially available under the names of Gafquat®734 and Gafquat®755.

Copolymers of vinyl pyrrolidone with methoimidazolinium chloride which are commercially available under the name of Luviquat®.

Quaternized polyvinyl alcohol.

The polymers with quaternary nitrogen atoms in the main polymer chain known by the names of Polyquaternium 2, Polyquaternium 17, Polyquaternium18 and Polyquaternium 27.

Cationic polymers from the first four groups mentioned are particularly preferred, Polyquaternium 2, Polyquaternium 10 and Polyquaternium 22 being most particularly preferred.

Other suitable conditioning agents are silicone oils, more particularly dialkyl and alkylaryl siloxanes, such as for example dimethyl polysiloxane and methylphenyl polysiloxane, and alkoxylated and quaternized analogs thereof. Examples of such silicones are the products marketed by Dow Corning under the names of DC 190, DC 200, DC 344, DC 345 and DC 1401 and the products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, quaternium-80).

Other suitable conditioning agents are paraffin oils, synthetically produced oligomeric alkenes and vegetable oils, such as jojoba oil, sunflower oil, orange oil, almond oil, wheatgerm oil and peach kernel oil.

Phospholipids, for example soya lecithin, egg lecithin and kephalins, are also suitable hair-conditioning compounds.

Other active substances, auxiliaries and additives are, for example,

nonionic polymers such as, for example, vinyl pyrrolidone/vinyl acrylate copolymers, polyvinyl pyrrolidone and vinyl pyrrolidone/vinyl acetate copolymers and polysiloxanes,

zwitterionic and amphoteric polymers such as, for example, acrylamido-propyl/trimethyl ammonium chloride/acrylate copolymers and octyl acrylamide/methyl methacrylate/tert.butyl aminoethyl methacrylate/2-hydroxypropyl methacrylate copolymers,

anionic polymers such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,

thickeners, such as agar agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as glucose and maleic acid,

protein hydrolyzates, more particularly elastin, collagen, keratin, milk protein, soya protein and wheat protein hydrolyzates, condensation products thereof with fatty acids and quaternized protein hydrolyzates,

perfume oils, dimethyl isosorbide and cyclodextrins,

solubilizers, such as ethanol, isopropanol, ethylene glycol, propylene glycol, glycerol and diethylene glycol,

antidandruff agents, such as piroctone olamine and zinc omadine,

other substances for adjusting the pH value,

active substances, such as panthenol, pantothenic acid, allantoin, pyrrolidone carboxylic acids and salts thereof, plant extracts and vitamins,

cholesterol,

sun protection factors,

consistency factors, such as sugar esters, polyol esters or polyol alkyl ethers,

fats and waxes, such as spermaceti, beeswax, montan wax, paraffins, fatty alcohols and fatty acid esters,

fatty acid alkanolamides,

complexing agents, such as EDTA, NTA and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex,

pearlizers, such as ethylene glycol mono- and distearate,

propellents, such as propane/butane mixtures, N₂O, dimethyl ether, CO₂ and air and

antioxidants.

To produce the colorants according to the invention, the constituents of the water-containing carrier are used in the usual quantities for this purpose. For example, emulsifiers are used in concentrations of 0.5 to 30% by weight while thickeners are used in concentrations of 0.1 to 25% by weight, based on the colorant as a whole.

The hair colorants according to the invention are normally adjusted to a mildly acidic or alkaline pH, i.e. to a pH of about 5 to 12. To this end, the colorants containing alkalizing agents, typically alkali metal or alkaline earth metal hydroxides, ammonia or organic amines. Preferred alkalizing agents are monoethanolamine, monoisopropanolamine, 2-amino-2-methylpropanol, 2-amino-2-methylpropane-1,3-diol, 2-amino-2-ethylpropane-1,3-diol, 2-amino-2-methylbutanol and triethanolamine and alkali metal and alkaline earth metal hydroxides. Within this group, monoethanolamine, triethanolamine and 2-amino-2-methylpropanol and 2-amino-2-methylpropane-1,3-diol are preferred. o-Amino acids, such as co-aminocaproic acid, may also be used as alkalizing agents.

In principle, the color can be oxidatively developed with atmospheric oxygen. However, a chemical oxidizing agent is preferably used, particularly when human hair is to be not only colored, but also lightened. Particularly suitable oxidizing agents are persulfates, chlorites and, in particular, hydrogen peroxide or addition products thereof with urea, melamine or sodium borate. Oxidation may also be carried out with enzymes. In this case, the enzymes may be used both to produce oxidizing per compounds and to enhance the effect of an oxidizing agent present in small quantities.

Thus, the enzymes (enzyme class 1: oxidoreductases) can transfer electrons from suitable primary intermediates (reducing agents) to atmospheric oxygen. Oxidases, such as tyrosinase and laccase, are preferred for this purpose, as are glucoseoxidase, uricase or pyruvate oxidase. Mention is also made of the procedure whereby the effect of small quantities (for example 1% and less, based on the formulation as a whole) of hydrogen peroxide is enhanced by peroxidases.

The preparation of the oxidizing agent is preferably mixed with the preparation of the oxidation dye precursors immediately before coloring of the hair. The ready-to-use hair coloring preparation formed should preferably have a pH value in the range from 6 to 12 and more particularly in the range from 7.5 to 10. In a particularly preferred embodiment, the hair colorant is used in a mildly alkaline medium. The application temperatures may be in the range from 15 to 40° C. and are preferably around the temperature of the scalp. After a contact time of about 5 to 45 minutes and more particularly 15 to 30 minutes, the hair colorant is removed from the hair to be colored by rinsing. There is no need for the hair to be washed with a shampoo where a carrier of high surfactant content, for example a coloring shampoo, has been used.

In the particular case of hair which is difficult to color, the preparation containing the oxidation dye precursors may be applied to the hair without preliminary mixing with the oxidation component. The oxidation component is applied after a contact time of 20 to 30 minutes, optionally after rinsing. After another contact time of 10 to 20 minutes, the hair is rinsed and, if desired, shampooed. In a first variant of this embodiment where the preliminary application of the dye precursors is intended to improve penetration into the hair, the corresponding formulation is adjusted to a pH value of about 4 to 7. In a second variant, oxidation with air is initially carried out, the formulation applied preferably having a pH value of 7 to 10. In the subsequent accelerated post-oxidation phase, it can be of advantage to use acidified peroxydisulfate solutions as the oxidizing agent.

Whichever of the processes mentioned above is used to apply the colorant according to the invention, development of the color may be supported and enhanced by adding certain metal ions to the colorant. Examples of such metal ions are Zn²⁺, Cu²⁺, Fe²⁺, Fe³⁺, Mn²⁺, Mn⁴⁺, Li⁺, Mg²⁺, Ca²⁺ and Al³⁺. Zn²⁺, Cu²⁺ and Mn²⁺ are particularly suitable. Basically, the metal ions may be used in the form of a physiologically compatible salt. Preferred salts are the acetates, sulfates, halides, lactates and tartrates. Development of the hair color can be accelerated and the color tone can be influenced as required through the use of these metal salts.

The present invention also relates to the use of the colorants described in the foregoing for coloring keratinous fibers.

The following Examples are intended to illustrate the invention.

### EXAMPLES

All quantities in the Examples are parts by weight.

1. Preparation of the coloring cream

| Mixture A | |
|---|---|
| Hydrenol ® D¹ | 8.50 g |
| Lorol ® techn.² | 2.00 g |
| Eumulgin ® B2³ | 0.75 g |
| Texapon ® NSO⁴ | 20.00 g |
| Dehyton ® K⁵ | 12.50 g |
| Water | 30.00 g |

¹C₁₆₋₁₈ fatty alcohol (INCI name: Cetearyl Alcohol) (HENKEL)
²C₁₂₋₁₈ fatty alcohol (INCI name: Coconut Alcohol) (HENKEL)
³cetylstearyl alcohol with ca. 20 EO units (INCI name: Ceteareth-20) (HENKEL)
⁴lauryl ether sulfate, sodium salt (ca. 27.5% active substance: INCI name: Sodium Laureth Sulfate) (HENKEL)
⁵N,N-dimethyl-N-(C₈₋₁₈ cocoamidopropyl)-ammonium acetobetaine (Ca. 30% active substance; INCI name: Aqua (Water), Cocamidopropyl Betaine) (HENKEL)

The substances Hydrenol D, Lorol and Eumulgin B2 were melted at 80° C., mixed with the water (heated to 80° C.) containing the Texapon NSO and Dehyton K and emulsified with vigorous stirring. The emulsion was then cooled twice with gentle stirring.

| Mixture B | |
|---|---|
| Sodium sulfite | 1.00 g |
| Ammonium sulfate | 1.00 g |
| Dye precursors | 2.5 mmol of each |
| Ammonia (25% solution) | to pH 10.0 |
| Water | 10.00 g |

The dye precursors were dissolved in the water (heated to 50° C.) while the sodium sulfite, ammonium sulfate and ammonia were added.

The dye precursor solution (mixture B) was added to the emulsion (mixture A) and, after adjustment to pH 10 with ammonia, the whole was made up with water to 100 parts by weight, followed by stirring until the temperature had reached room temperature.

2. Coloring of the Keratinous Fibers

The coloring cream thus obtained was mixed with a 3% H₂O₂ solution in a ratio of 2:1 and the mixture was applied to 5 cm long tresses of standardized, 80% gray but not specially pretreated human hair (Kerling). After a contact time of 30 minutes, the hair was rinsed, washed with a standard shampoo and then dried.

The results of the coloring tests were evaluated on the basis of the Taschenlexikon für Farben (A. Kornerup, J. H. Wanscher, Muster-Schmidt Verlag, 1961) and are set out in Table I. The following abbreviations were used.

| | |
|---|---|
| K1: | 1-methyl-2-methoxy-3,5-diaminobenzene |
| K2: | 3-amino-2-chloro-6-methylphenol |
| K3: | 5-amino-4-chloro-2-methylphenol |
| K4: | 5-(2′-hydroxyethyl)-amino-2-methylphenol |
| K5: | 5-amino-2-methylphenol |
| K6: | 2-amino-3-hydroxypyridine |
| K7: | 1,3-bis-(2,4-diaminophenoxy)-propane |
| K8: | 3-amino-2-methylamino-6-methoxypyridine |
| K9: | 2-methylresorcinol |
| K10: | 2,4-diaminophenoxyethanol |
| K11: | resorcinol |
| K12: | 3,5-diamino-2,6-dimethoxypyridine |
| K13: | 1,5-dihydroxynaphthalene |
| K14: | m-aminophenol |
| E1: | 2-(β-hydroxyethyl)-p-phenylenediamine sulfate |
| E2: | p-toluylenediamine sulfate |
| E3: | p-phenylenediamine |
| E4: | N,N-bis-(2′-hydroxyethyl)-p-phenylenediamine sulfate |
| E5: | N,N′-bis-(4′-aminophenyl)-1,4-diazacycloheptane chloride |
| E6: | 4,5-diamino-1-(β-hydroxyethyl)-pyrazole sulfate |
| E7: | p-aminophenol |
| E8: | bis-(2-hydroxy-5-aminophenyl)-methane |
| E9: | 4-amino-2-aminomethylphenol |
| E10: | 4-amino-3-methylphenol |

| Secondary | Second | | |
|---|---|---|---|
| intermediate | secondary | Primary | |
| of formula (I) | intermediate | intermediate | Color |
| K1 | K2 | E1 | Dark magenta 13F6 |
| K1 | K3 | E1 | Lake red 8C8 |
| K1 | K4 | E1 | Brown-red 10D6 |
| K1 | K5 | E1 | Violet-brown 10E8 |
| K1 | K6 | E2 | Lake red 9C8 |
| K1 | K7 | E2 | Dark violet 17F4 |
| K1 | K8 | E2 | Dark brown 7F7 |
| K1 | K9 | E3 | Tomato red 8C8 |
| K1 | K10 | E3 | Dark violet 17F4 |
| K1 | K5 | E4 | Dark magenta 13F5 |
| K1 | K11 | E4 | Madder red 9A7 |
| K1 | K14 | E4 | Brown-red 10D6 |
| K1 | K4 | E5 | Gray-ruby 12C5 |
| K1 | K6 | E6 | Dark violet 15F8 |
| K1 | K4 | E6 | Violet-brown 10E7 |
| K1 | K3 | E7 | Red-gold 6C7 |
| K1 | K10 | E8 | Garnet-brown 9D8 |
| K1 | K8 | E8 | Cocoa brown 6E6 |
| K1 | K6 | E8 | Brown-orange 7C6 |
| K1 | K8 | E9 | Cocoa brown 6E6 |
| K1 | K12 | E10 | Light brown 7D5 |
| K1 | K6 | E10 | Gray-orange 5B4 |
| K1 | K13 | E10 | Flat red 8C4 |

What is claimed is:

1. An oxidation colorant composition for coloring keratin fibers comprising, in a medium suitable for coloring:
(a) at least one first secondary intermediate comprising at least one m-phenylenediamine derivative compound corresponding to formula (I) or a physiologically compatible salt thereof: 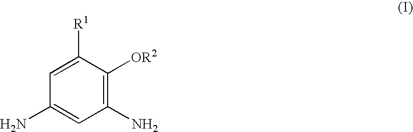
wherein R¹ is a branched or unbranched C₁₋₈ alkyl group and R² is a branched or unbranched C₁₋₈ alkyl group or a phenyl group, the phenyl group being optionally substituted by one or more C₁₋₄ alkyl groups or by one or more halogen atoms;
(b) at least one second secondary intermediate selected from 2-amino-3-hydroxy-5-chloropyridine, 2,6-dihydroxy-3,4-dimethylpyridine, 2,6-bis-(β-hydroxyethylamino)-toluene, 2,4-dichloro-3-aminophenol, 3-amino-2-chloro-6-methylphenol, 5-amino-4-chloro-2-methylphenol, 5-(2′-hydroxyethyl)-amino-2-methylphenol, 5-amino-2-methylphenol, 2-amino-3-hydroxypyridine, 3-amino-2-methylamino-6-methoxypyridine, 2-methylresorcinol, 2,4-diaminophenoxyethanol, 1,3-bis-(2,4-diaminophenoxy)-propane, resorcinol, m-aminophenol, 3,5-diamino-2,6-dimethoxypyridine, 1,7-dihydroxynaphthalene, 2,7-dihydroxynaphthalene, 1,5-dihydroxy-naphthalene, 4-hydroxyindole or 6-hydroxyindole, or combinations thereof; and
(c) at least one primary intermediate.

2. The composition of claim 1, wherein the compound corresponding to formula (I) comprises 1-methyl-2-methoxy-3,5-diaminobenzene or a physiologically compatible salt thereof.

3. The composition of claim 1, wherein the second secondary intermediate is selected from 3-amino-2-chloro-6-methylphenol, 5-amino-4-chloro-2-methylphenol, 5-(2′-hydroxyethyl)-amino-2-methylphenol, 5-amino-2-methylphenol, 2-amino-3-hydroxypyridine, 3-amino-2-methylamino-6-methoxypyridine, 2-methylresorcinol, 2,4-diaminophenoxyethanol, 1,3-bis-(2,4-diaminophenoxy)-propane, resorcinol, m-aminophenol, 3,5-diamino-2,6-dimethoxypyridine or 1,5-dihydroxynaphthalene, or combinations thereof.

4. The composition of claim 3, wherein the second secondary intermediate is selected from 3-amino-2-chloro-6-methylphenol, 5-amino-4-chloro-2-methylphenol, 2-amino-3-hydroxypyridine, 3-amino-2-methylamino-6-methoxypyridine, 2,4-diaminophenoxyethanol, 3,5-diamino-2,6-dimethoxypyridine or 1,5-dihydroxynaphthalene, or combinations thereof.

5. The composition of claim 1, wherein the primary intermediate comprises at least one p-phenylenediamine derivative.

6. The composition of claim 5, wherein the p-phenylenediamine derivative comprises p-phenylenediamine, p-toluylenediamine, 2-(β-hydroxyethyl)-p-phenylenediamine or N,N-bis-(2-hydroxyethyl)-p-phenylenediamine, or a physiologically compatible salt thereof, or combinations thereof.

7. The composition of claim 1, wherein the primary intermediate comprises at least one binuclear primary intermediate.

8. The composition of claim 7, wherein the binuclear primary intermediate comprises bis-(2-hydroxy-5-aminophenyl)-methane or a physiologically compatible salt thereof.

9. The composition of claim 1, wherein the primary intermediate comprises at least one p-aminophenol derivative.

10. The composition of claim 9, wherein the p-aminophenol derivative comprises p-aminophenol, 4-amino-3-methylphenol, 4-amino-2-aminomethyl phenol or 4-amino-2-(diethylamino)-methyl)-phenol, or a physiologically compatible salt thereof, or combinations thereof.

11. The composition of claim 1, wherein the primary intermediate comprises at least one heterocycle selected from a pyridine derivative, a pyrimidine derivative, a pyrazole derivative or a pyrazole/pyrimidine derivative, or a physiologically compatible salt thereof, or combinations thereof.

12. The composition of claim 1 further comprising at least one substantive dye.

13. The composition of claim 1, wherein the composition has a pH ranging from 7.5 to 10.

14. The composition of claim 1 wherein the first secondary intermediate comprises 1-methyl-2-methoxy-3,5-diaminobenzene or a physiologically salt thereof, and the second secondary intermediate is selected from 3-amino-2-chloro-6-methylphenol, 5-amino-4-chloro-2-methylphenol, 2-amino-3-hydroxypyridine, 3-amino-2-methylamino-6-methoxypyridine, 2,4-diaminophenoxyethanol, 3,5-diamino-2,6-dimethoxypyridine or 1,5-dihydroxynaphthalene, or combinations thereof.

15. The composition of claim 14 wherein the primary intermediate comprises a p-phenylenediamine derivative selected from p-phenylenediamine, p-toluylenediamine, 2-(β-hydroxyethyl)-p-phenylene-diamine or N,N-bis-(2-hydroxyethyl)-p-phenylenediamine, a p-aminophenol derivative selected from p-aminophenol, 4-amino-3-methylphenol, 4-amino-2-aminomethyl phenol or 4-amino-2-(diethylamino)-methyl)-phenol, or a binuclear primary intermediate selected from bis-(2-hydroxy-5-aminophenyl)methane, or a physiologically compatible salt thereof, or combinations thereof.

16. A method of coloring keratinous fibers comprising applying to keratinous fibers the oxidation colorant composition of claim 1.

17. The method of claim 16, wherein the compound corresponding to formula (I) comprises 1-methyl-2-methoxy-3,5-diaminobenzene or a physiologically compatible salt thereof.

18. The method of claim 17, wherein the second secondary intermediate is selected from 3-amino-2-chloro-6-methylphenol, 5-amino-4-chloro-2-methylphenol, 5-(2′-hydroxyethyl)-amino-2-methylphenol, 5-amino-2-methylphenol, 2-amino-3-hydroxypyridine, 3-amino-2-methylamino-6-methoxypyridine, 2-methylresorcinol, 2,4-diaminophenoxyethanol, 1,3-bis-(2,4-diaminophenoxy)-propane, resorcinol, m-aminophenol, 3,5-diamino-2,6-dimethoxypyridine or 1,5-dihydroxynaphthalene, or combinations thereof.

19. The method of claim 18 wherein the primary intermediate comprises a p-phenylenediamine derivative, a p-aminophenol derivative, or a binuclear primary intermediate or combinations thereof.
